(19) United States
(12) Patent Application Publication
(10) Pub. No.: US 2008/0312516 A1
(43) Pub. Date: Dec. 18, 2008

(54) Living body information detection and display method

(75) Inventors: Noriyuki Ozaki, Kariya-city (JP); Hiroto Nakatani, Nagoya-city (JP); Kenichi Yanai, Nisshin-city (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 12/155,495

(22) Filed: Jun. 5, 2008

Related U.S. Application Data
(63) Continuation of application No. 10/952,874, filed on Sep. 30, 2004, now abandoned.

(30) Foreign Application Priority Data
Nov. 19, 2003 (JP) 2003-389527

(51) Int. Cl. A61B 5/00

(52) U.S. Cl. 600/301

(57) ABSTRACT

A living body information display apparatus, together with a sleeping posture and position detection apparatus, is used to correctly and suitably capture an abnormal condition in a living body while the living body is sleeping/lying on a bed or on the apparatus. Living body information and sleeping posture/position are captured by pressure sensors placed under a sleeper. The living body information includes, for example, respiratory information, body movement information, and sleeping posture. The information of the sleeper is represented in color-coded time chart, in writing and in a rough image based on the sensor signals. By using this apparatus, a user can readily understands the position, posture and respiratory information of the sleeper.

### CROSS REFERENCE TO RELATED APPLICATIONS

This application is a continuation of U.S. application Ser. No. 10/952,874, which was filed on Sep. 30, 2004. This application incorporates the contents of U.S. application Ser. No. 10/952,874 by reference.

### BACKGROUND OF THE INVENTION

The present invention relates to a living body information display apparatus and method and the like.

It is widely known that there exists an apparatus that detects and takes measurement of apnea and/or hypopnea of a sleeper. For example, JP-A-8-131421 (with special notices of FIG. 12, 15, 18, 22) describes the results of measurements taken by one such apparatus. The apparatus disclosed in JP-A-8-131421 displays the following pairs of information in an arranged manner: i) an index of apnea of an examinee in a sleeping condition and a degree of oxygen saturation, ii) the index of apnea and a sleeping posture (i.e., whether the examinee is on his/her right side, his/her left side, or his/her backside), iii) the index of apnea and a body movement, and iv) the index of apnea and a sound level of snoring.

Specifically, sleeping posture is derived from respiration information that is sensed by vibration detecting respiration sensors placed at the center, left, and right sides of the bed. Generally, when the center sensors yield periodic respiration signals and the left sensors yield non-periodic signals, caused by, for example, body movement, the living body is determined to be lying sideways on his/her right side. Alternatively, if the right sensors yield non-periodic signals caused by, for example, body movement, and the center sensors yield periodic respiration signals, the living body is determined to be on his/her left side. Also, when only the center sensors yield periodic respiration signals, the living body is determined to be lying on his/her backside.

However, the mere determination and display of an examinee's sleeping posture does not lead to an intuitive grasp of the sleeping posture of an examinee suffering from a respiratory abnormality such as apnea syndrome and the like. For example, the relative position of an examinee's limbs effects the respiratory system of an examinee lying on his/her backside. Similarly, the relative curvature of the examinee's back affects the respiratory system of an examinee lying on either of his/her sides. Also, vibrant movement of the examinee on the bed while sleeping will result in an inaccurate determination. For example, when an examinee rotates toward lying with his/her feet on the headboard side of the bed, the center and the side sensors yield incorrect respiration signals at the point where the examinee is lying sideways. To be precise in terms of sleeping posture, the sideways position means, in this description, that the examinee's body is rotated 90 degrees from the normal, supine lying position on the bed. The ‘normal’ position of the examinee means that he/she is lying supine with his/her head at the headboard side and his/her feet extended opposite therefrom with his/her spine placed in parallel with the longer side of the bed.

Respiratory information is described herein as an example of living body information. However, when a certain abnormality is observed, other types of living body information, coupled with an accurately captured sleeping posture, could also be utilized to effectively identify the cause of a problem. In other words, in addition to identifying the basic sleeping posture as being sideways, face up, or face down, the examiner may capture the actual sleeping posture at the occurrence of a respiratory abnormality. The abnormality may then be diagnosed with higher certainty to be the result of either an awkward sleeping posture when the sleeping position is different from a normal one or some other probable cause when the sleeping posture is substantially normal.

### SUMMARY OF THE INVENTION

In view of the foregoing problems, an object of the present invention is to provide a living body information display apparatus that displays a more accurately captured sleeping posture at the occurrence of an abnormality. Another object of the invention is to provide a suitable sleeping posture and position detection apparatus that can be used with the living body information display apparatus.

The living body information display apparatus to achieve the first object stated above comprises sensors, a living body information detection means, and a display controlling means. The sensors are placed under a sleeper in ‘rows’ and ‘columns’ to detect pressure and vibration signals created by the sleeper. The living body information, such as respiration, body movement, and sleeping posture/position, is detected and displayed by using the living body information detection means and the display control means. The posture and position of the sleeper is, together with the living body information such as respiration, displayed with intuitively understandable visuals (in figures and graphs) across the same period of time, thus leading to an easy determination of abnormality (refer to FIG. 1, for example).

The sleeping posture and position detection apparatus to achieve the second object of the present invention comprises the same components as the first one, that is, sensors, a sleeping posture detection means, and a position determination means. In this case, however, the signals from those sensors are processed differently to retrieve the desired information.

Sensors are placed under a sleeper in directions that are approximately vertical and parallel to the sleeper, similar to ‘rows’ and ‘columns’ having predetermined spacing. The sensors output signals based on pressure and vibration created by the sleeper. The sleeping posture detection means detects areas of pressure and/or vibration, as well as a sleeping posture based on the pressure and/or vibration-related signals outputted from the sensors. The position determination means determines the position of the examinee as being supine/prone or sideways. This determination is based on the pressure or vibration-related signals outputted from the sensors and the change of pressure and/or vibration across each ‘row.’ This is because the human body is generally wider than it is thick. Therefore, the change rate of the pressure and/or vibration values are inevitably different between the cases where the examinee is in the supine/prone position and where the examinee is in the sideways position. Namely, when the examinee is in the supine/prone position, the change rate of the pressure and/or vibration values are relatively gradual. When the examinee is in the sideways position, the change rate of the pressure and/or vibration values are relatively steep. For example, see the bodies represented in graphical form in FIGS. 9A and 9B. This change rate indicates whether the examinee is in the supine/prone position or the sideways position.

One method of determining an examinee's position could be done in the following way. When a difference between a maximum value of pressure or vibration and a corresponding value (either pressure or vibration) detected at a position located at a predetermined distance from where the maximum value is measured is less than a predetermined value, the examinee is determined to be in the supine/prone position. When the value is more than the predetermined value, the examinee is determined to be in the sideways position. However, the body of the examinee may be twisted. In one position the body is almost in the supine/prone position, but a portion of the body is in the sideways position. In another position, the body is almost in the sideways position, but a portion of the body is in the supine/prone position. In either of these cases, the sensors in a certain row could output erroneous pressure or vibration signals. To prevent an inaccurate body position calculation, signals from multiple rows of sensors must be utilized in the following way. First, differences are calculated along a row between a maximum value of pressure or vibration, and a corresponding value detected at a position located a (either pressure or vibration value) predetermined distance from where the maximum value is measured. If an average of differences in multiple rows is less than the predetermined value, the examinee is determined to be in the supine/prone position. If the average is more than the predetermined value, the examinee is determined to be in the sideways position. The position of the body of the examinee can be determined more accurately in this manner.

An alternative method of determining an examinee's position is now proposed. The position is determined to be supine/prone or sideways by analyzing the pressure and/or vibration signals outputted from the sensors, the area of the pressure and vibration, and the change rate of the maximum value of pressure or vibration over a different period of time. As described above, a generic human body is wider than it is thick and, thus, the area it occupies (area of contact between the body and the bed pad) is different whether the body is lying on its side or on its back. Therefore, a change in the area where pressure or vibration is applied can be used as an indicator of transition from the supine/prone position to the sideways position, or vice versa. However, there is a possibility of an inaccurate determination if it is based only on this condition. Therefore, the present invention also utilizes the change rate of the maximum value of pressure or vibration. When the position of the body changes from supine/prone to sideways, the pressure or vibration per unit area beneath the body must increase, and, thus the maximum value of pressure or vibration must increase. On the contrary, when the position of the body is changed from sideways to supine/prone, the pressure or vibration per unit area decreases and, thus, the maximum value of pressure or vibration decreases accordingly. In this manner, body position can be accurately determined based on the area to which pressure or vibration is applied or based on the change rate of the maximum value of pressure or vibration.

### BRIEF DESCRIPTION OF THE DRAWINGS

While the appended claims set forth the features of the present invention with particularity, the invention together with its objects and advantages, may be best understood from the following detailed description taken in conjunction with the accompanying drawings, in which:

FIG. 1 illustrates one embodiment of a display according to a living body information display apparatus of the present invention;

FIG. 2 is a plan view of the living body information display apparatus of the present invention;

FIG. 3 is a block diagram of a circuit of a control section of the living body information display apparatus of the present invention;

FIG. 4 is a perspective view of the living body information display apparatus of the present invention installed on a bed;

FIG. 5 is a flow chart of a living body position detection process according to the living body information display of the present invention;

FIG. 6 is a flow chart of a first embodiment of a body movement determination process according to the present invention;

FIG. 7 is a flow chart of a slight movement determination process according to the present invention;

FIG. 8 is a flow chart of a sleeping posture determination process according to the present invention;

FIG. 9A is a graph illustrating a cross-sectional view of a torso of a sleeper lying in a supine/prone position on the living body information display apparatus of the present invention;

FIG. 9B is a graph illustrating a cross-sectional view of a torso of a sleeper lying in a sideways position on the living body information display apparatus of the present invention;

FIG. 10 illustrates a second embodiment of a display of the living body information display apparatus of the present invention; and

FIG. 11 is a flow chart of a second embodiment of the sleeping posture determination process of the present invention.

### DETAILED DESCRIPTION OF THE PREFERRED EMBODIMENT

A preferred embodiment of the present invention is described herein with reference to the drawings. Furthermore, the present invention shall not be limited to the following examples but shall include various forms that fall within the scope of the art.

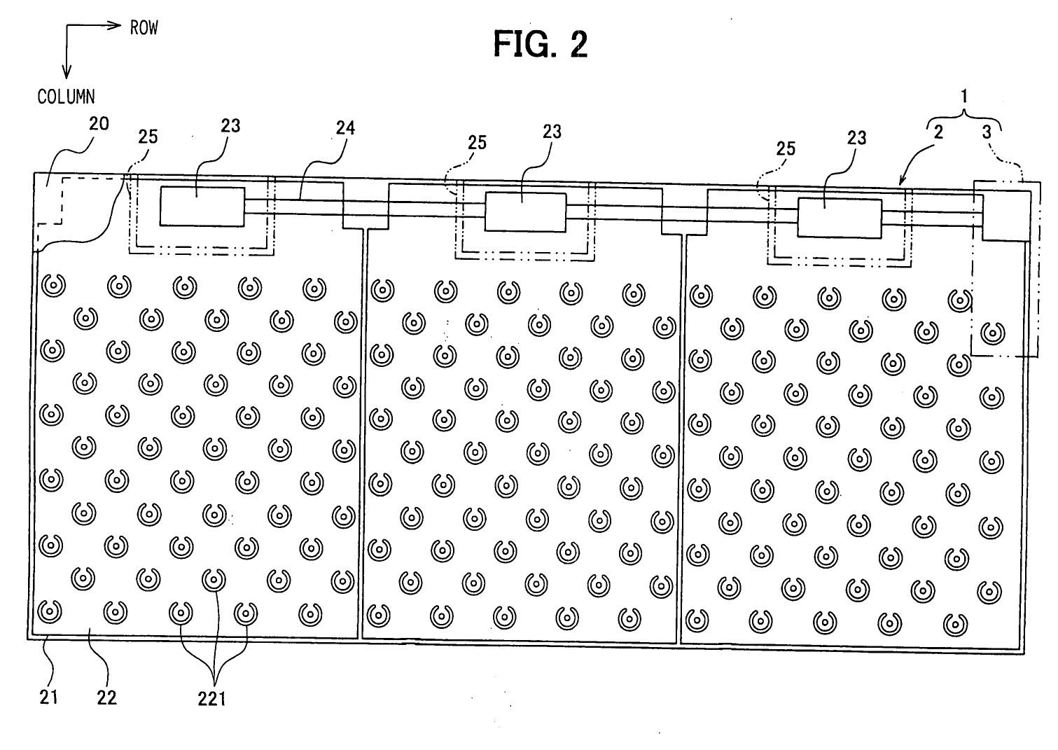

FIG. 2 is a plan view of a living body information display apparatus 1 of an embodiment of the present invention. The living body information display apparatus 1 comprises a sensor sheet 2 and controller 3. The controller 3 is attached to the edge (right shoulder of the front view in this embodiment) of the rectangular sensor sheet 2. The living body information display apparatus 1 is used on a bed 50, as shown in FIG. 4. The bed 50 comprises a lying section 51 that carries bedding 60, such as a bed pad and the like, and a headboard 52 that is vertically attached to the lying section 51. The living body information display apparatus 1 is disposed under the bedding 60 on the lying section 51 of the bed 50.

The living body information display apparatus 1 is placed on the headboard side of the center of the lying section 51 to be beneath the torso of a sleeper lying on the bed 50.

First, the sensor sheet 2 is described. The sensor sheet 2 consists of multiple layers. From top to bottom, the layers include an upper PU film 20, a pressure sensor layer 22, a PVC sheet 26, and a lower PU film 21.

The upper PU film 20 and the lower PU film 21 are made of soft and transparent polyurethane resin films. The upper PU film 20 and the lower PU film 21 have the same rectangular shape and size as the sensor sheet 2 and the four sides of those sheets are connected to each other. As a result, the pressure sensor layer 22 and the PVC sheet 26 are disposed therein and protected from the atmosphere outside.

Three pressure sensor layers 22 are placed inside the rectangular sensor sheet 2. The pressure sensor layers 22 are positioned adjacent to each other at equally divided portions along the longer side of the sensor sheet. Each of the three pressure sensor layers 22 have the same structure. Each includes fifty-five regularly arranged pressure-sensing devices 221 comprising a “sensor.” The pressure-sensing devices 221 change their resistance according to the applied pressure. Therefore, there are 165 (55 multiplied by 3) pressure-sensing devices 221 in the whole sensor sheet 2. More specifically, there are ten rows of pressure-sensing devices 221 that are perpendicular to the longer side of the sensor sheet 2. The rows each include either 5 sensors or 6 sensors and are arranged in an alternating manner. Every pressure sensor layer 22 has the same sensor arrangement such that the pattern is maintained even where two sensor layers 22 meet. Therefore, when one of the edges of two adjacent sensor layers 22 has 6 sensing devices, the other has 5 sensing devices to maintain the above-described alternating arrangement. Also, a rubber pad (not shown) is fixed with adhesive or glue or the like on the upper surface of each pressure-sensing device 221.

The sensor sheet 2 of the present embodiment is shown in FIG. 4 as being used with its longer side in the width direction of the bed 50. That means the height direction of the sleeper lying on the bed 50 is perpendicular to the direction of longer side of the sensor sheet 2. For purposes of discussion, the pressure-sensing devices 221 on the sensor layers 22 that are arranged perpendicular to the longer side of the bed are defined as ‘rows.’ Each sensor layer 22 includes 5 pressure-sensing devices per ‘row.’ The pressure-sensing devices 221 that are arranged parallel to the longer side of the sensor sheet 2 are defined as ‘columns.’ Each sensor layer 22 includes 10 ‘columns’ having alternating 6 sensors and 5 sensors, as described above.

Furthermore, a sensor selection section 23, instead of pressure-sensing devices 221, is positioned on the pressure sensor layer 22 in a certain area near the headboard 52 side of the sensor sheet 2 when the sheet 2 is placed on the lying section 51 of the bed 50. The sensor selection sections 23 on each of the three pressure sensor layers 22 are connected to each other via a film type circuit 24. As shown in FIG. 2, the right-most sensor selection section 23 is connected to the controller 3. Although FIG. 2 does not explicitly show a circuit electrically connecting each pressure-sensing device 221 to the sensor selection sections 23, an applied pressure to each of the pressure-sensing device 221 can independently be detected. This detection is based on a drop in voltage across each pressure-sensing device 221 because the resistance of each pressure-sensing device 221 varies according to the pressure applied.

Furthermore, the upper PU film 20 has a maintenance hole 25 that can be opened and closed near each of the sensor selection sections 23. More concretely, the maintenance holes 25 are formed slightly larger than the sensor selection sections 23 and are covered by the upper PU film 20. The upper PU film 20 is slightly larger than the maintenance holes 25 and can be opened/closed at will. In this manner, convenience of maintenance of the sensor selection sections 23 and the film type circuit 24 connecting the sensors to the circuit is improved.

The PVC sheet 26 is a hard polyvinyl chloride resin sheet. The PVC sheet 26 has the same shape as the pressure sensor layer 22 and includes three portions arranged in a row along the longer side on the rectangular sensor sheet 2. The following characteristics should be considered when selecting a hardness of the PVC sheet 26. The sensor sheet 2 is used on the bed 50 and the condition of the bedding may affect the sensitivity of the pressure-sensing devices 221. In other words, when the bedding is soft the pressure-sensing devices 221 may not be properly supported to detect an accurate pressure signal from the sleeper. Thus, the PVC sheet 26 compensates for the flexibility of the bedding to reduce uneven sinking/giving-in of the pressure-sensing devices 221 therein. This also reduces any delayed response by the pressure-sensing devices 221 to a change of pressure. If sinking/giving-in of the pressure-sensing device 221 has to be suppressed, a very hard PVC sheet 26 should be used. But that makes the bed 50 very uncomfortable. Thus, the hardness of the PVC sheet 26 has to be balanced between the sensitivity to pressure changes and sleeping comfort within the range of tolerance of the unevenness of pressure change.

While the upper PU film 20 and the lower PU film 21 have been disclosed as being polyurethane films and the PVC sheet 26 is disclosed as being a polyvinyl chloride resin sheet, the films and sheet are not restricted to those materials. The films and sheet may be made of any other resin film or sheet or any non-resin film or sheet.

An advantage of the above-described structure including the upper PU film 20, the pressure sensor layer 22, the PVC sheet 26, and the lower PU film 21, and the rectangular sensor sheet 2 is that it can be folded into one-third of its original size. The structure folds at two seams where only the upper and lower PU films 20, 21 and the film-type circuit 24 exist. This eliminates any troubles with the pressure sensor layers 22 and the PVC sheet 26. The upper and lower PU films 20, 21 are attached to each other and the sensor sheet 2 is made foldable at the attached portions. Furthermore, the film type circuit 24 is made of a fold-tolerant material in order to avoid problems. When the sensor sheet 2 is folded, two surfaces of the upper PU films 20 are placed against each other. However, since the pressure-sensing devices 221 are arranged in an alternating manner, as described above, there is no occasion for the rubber pads on the pressure-sensing devices 221 to interfere with each other.

Next, the controller 3 is described.

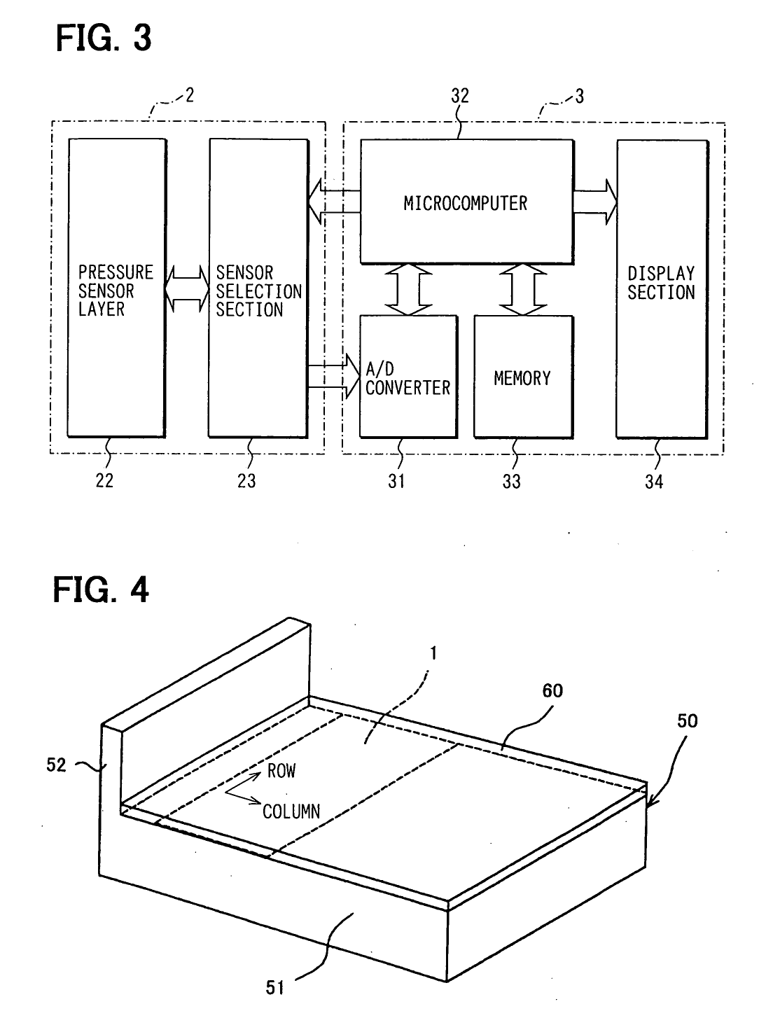

The controller 3 comprises, as shown in FIG. 3, an A/D converter 31, a microcomputer 32, a memory 33, and a display section 34. The controller 3 controls selection of the pressure-sensing devices 221 in the pressure sensor layers 22 chosen by the sensor selection section 23 successively. The controller 3 then sends a pressure signal (hereinafter referred to as an AD value), that is converted to digital from analog by the A/D converter 31 to the microcomputer 32. Then, the microcomputer 32 sends a switching signal to the sensor selection section 23 to switch the pressure signal to be inputted. By continuously repeating the above procedure, the microcomputer 32 periodically collects pressure signals from all of the pressure-sensing devices 221 and stores them in the memory 33.

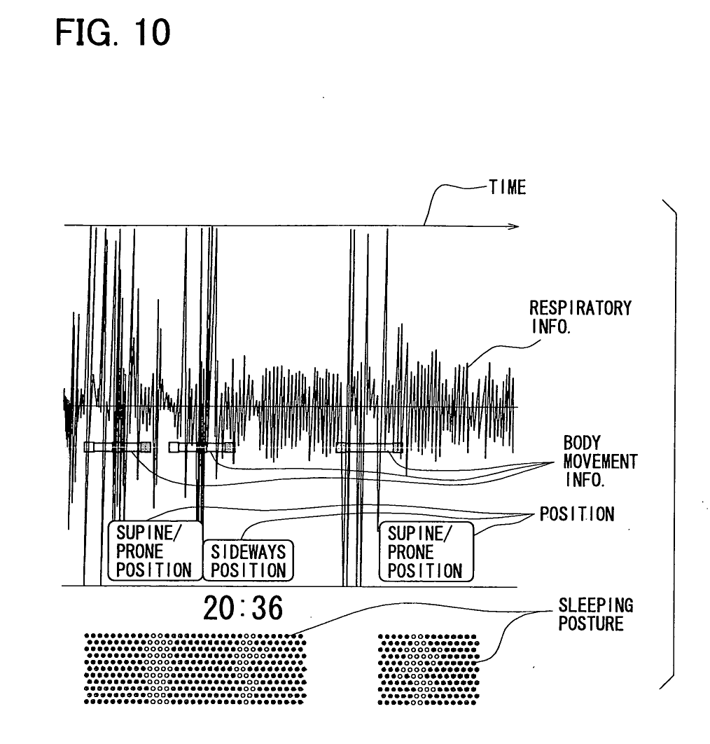

Once the pressure signals are all stored in the memory 33, the microcomputer 32, based on the pressure signals, executes a certain processing program to generate respiratory curves. The microcomputer 32 then outputs the number of occurrences and the time of occurrences of apnea and hypopnea to the display section 34 according to the respiratory curve. The microcomputer 32 also outputs body movement information to the display section 34 based on the occurrences of body movement and slight movement. Alternatively, the microcomputer 32 may output posture information and sleeping posture to the display section 34. These kinds of information are displayed across a period of time, as shown in FIG. 10.

In the present embodiment, just as the controller 3 is integrated with the sensor sheet 2, the display section 34 is integrated into the controller 3. However, the controller 3 may be separate from the sensor sheet 2 and connected to it by signal wiring. In that manner, a personal computer or the like may substitute the controller 3. This relaxes any restrictions on the size of the display section 34 and allows for a larger display compared to the integrated type of structure.

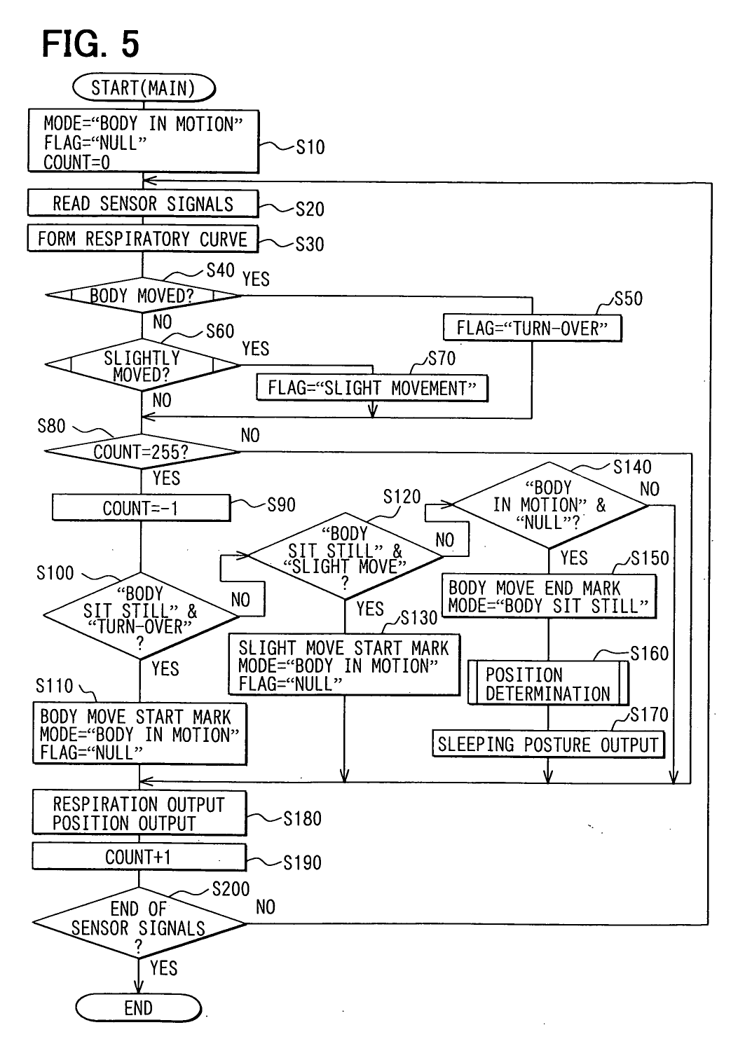

Now, the operation of the living body information display apparatus 1 in the present embodiment is described referring to the FIGS. 5 to 10.

FIG. 5 is a flow chart illustrating an entire process conducted by a controller 3 of the living body information display apparatus 1.

First, the controller 3 sets a mode to ‘body in motion,’ a flag to ‘NULL,’ and a data count to ‘0’ (zero) at (step S10). The controller 3 then reads sensor signals from the sensors (step S20). The controller 3 then generates a respiratory curve (step S30) and determines if body movement has occurred (step S40). If the controller 3 determines that body movement has occurred, it sets the flag to ‘TURN-OVER’ (step S50) and proceeds to step S80.

Alternatively, if the controller 3 determines that body movement has not occurred, it checks to see if slight movement has occurred (step S60). If the controller 3 determines that slight movement has occurred, then it sets the flag to ‘SLIGHT MOVEMENT’ and proceeds to step S80. However, if the controller 3 determines that slight movement has not occurred, it simply proceeds to step S80.

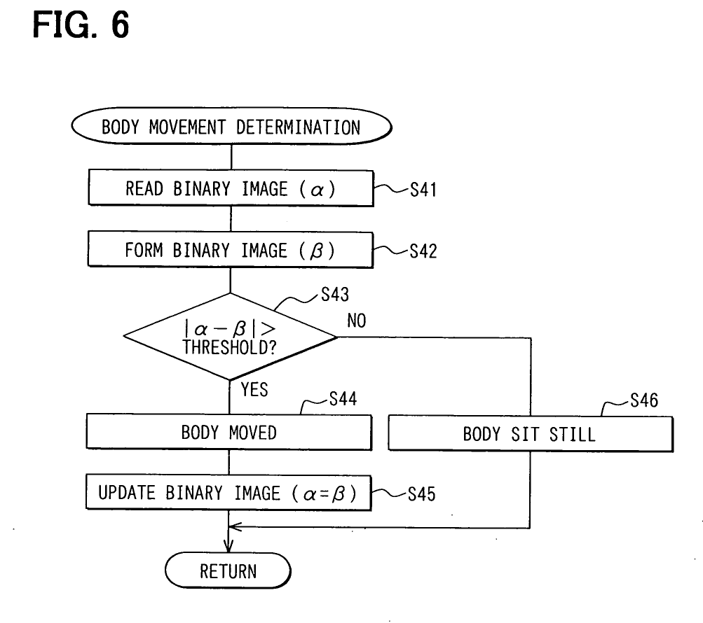

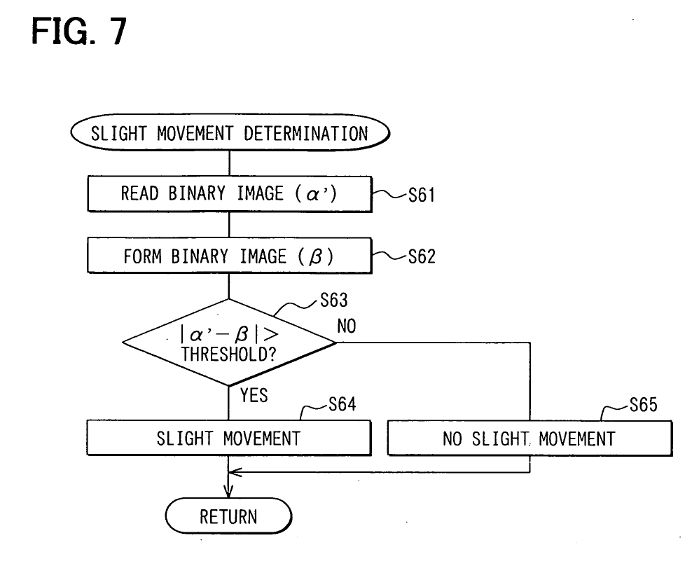

The processes of determining body movement (step S40) and slight movement (step S60) will now be described in more detail with reference to FIGS. 6 and 7, respectively.

FIG. 6 is a flowchart illustrating the body movement determination process provided in step S40. The controller 3 first reads a binary image of pressure distribution (α), which was formed by using signals received from each of the pressure-sensing devices 221 that have detected a value above a predetermined value, from the memory 33. That is, this binary image (α) shows the distribution of pressure applied by the sleeper to the bedding 10 and the like. It should be appreciated that this stored binary image (α) is updated whenever body movement such as a turnover occurs. The process of updating occurs at step S45, which will be described in more detail later.

Next, the controller compares the latest sensor signals from each pressure-sensing device 221 to the predetermined value to form a binary image of pressure distribution (β) at step S42.

Then, the controller 3 determines whether there was a change in pressure distribution at each pressure-sensing device 221 (step S43). This is determined by comparing the number of pressure sensors 221 detecting pressure in the past binary image (α) with the number of pressure sensors 221 detecting pressure in the present binary image (α). If the controller 3 determines that a difference between the number of the sensors 221 detecting pressure in the two images (α, β) is greater than a predetermined number, it identifies that a change in position has occurred. Furthermore, if the controller 3 determines that a positional gap, which is also represented by a number, in the pressure-sensing devices 221 detecting pressure between the two images (α, β) is above a certain number, it identifies that a change in position has occurred.

Therefore, when the controller 3 identifies a difference between the past and the present binary images (α, β) (step S43:YES) it determines that body movement has occurred (step S44). Additionally, the controller 3 overwrites the past binary image (α) with the present binary image (β) and stores the updated binary image (α=β) in the memory 33 (step S45). On the contrary, when the controller 3 identifies no difference between the images (α, β) (step S43:NO), it determines that no body movement has occurred (step S46).

Now, the process of step S60 including determining slight movement is described with reference to FIG. 7.

First, the controller 3 reads a binary image of pressure distribution (α′) from the memory 33 (step S61). The binary image (α′) is one that is constructed at the beginning or top of a 256-cycle period. The 256-cycle period is defined by the total number of signals each of the pressure-sensing devices 221 send to the controller 3 per process period. Therefore, the stored binary image (α′) is referred to hereinafter as a top-of-the-series binary image. It should be understood that slight movement is rather local compared to body movement and, therefore, binary images must be compared more frequently during the slight movement determination than during the body movement determination described above. In the present embodiment, the binary image is updated during each of the 256 cycles (corresponding to every 25.6 seconds in the present embodiment) and a top-of-the-series binary image of pressure distribution is created and stored as a sample to be retrieved.

Next, the controller 3 compares the latest signal from each of the pressure-sensing devices 221 to the predetermined value. This provides a visualization of the pressure distribution in the form of a binary image (β) (step S62). It should be appreciated that this is the same process described with reference to step S42 of FIG. 6.

Next, the controller derives the pressure distribution binary image (α′) at the top of 256 cycles (step S61) and compares it to the present binary image (β). This comparison enables the controller to determine whether there was a change in the pressure distribution based on each of the pressure-sensing devices 221 (step S63). It should be appreciated that this determination method is substantially the same as the one described above with reference to step S43 of FIG. 6, with an exception to the magnitude of the threshold. The threshold to determine a change in pressure distribution during the slight movement determination process is relatively small compared to the threshold used in the body movement determination of FIG. 6.

Nevertheless, if the controller 3 identifies a difference between the pressure distribution binary image (α′) generated at the top of the 256 cycles and the present pressure distribution binary image (β) (step S63:YES), it determines slight movement has occurred (step S64). If the controller 3 identifies no difference (step S63:No), it determines that no slight movement has occurred (step S65).

The above description regards only the body movement determination in step S40 and the slight movement determination in step S60. Now the description refers back to step S80 of FIG. 5.

In step S80, the controller 3 determines whether the data count equals 255. This is because body movement starts and ends gradually over a certain period of time. That is, a certain period of time must be taken as a grace period to make sure that body movement really occurred. Therefore, the controller 3 of the present embodiment waits 256 cycles (25.6 seconds) before displaying an image. If the data count is equal to 255 (step S80:YES), the controller 3 reduces the data count by 1 (step S90) and proceeds to step S100.

In step S100, the controller 3 determines whether the mode is set to ‘body sit still’ and the flag is set to ‘TURN-OVER.’ If the controller 3 determines that each of the above conditions are satisfied (step S100:YES), it displays a body movement starting sign on the display section 34. Simultaneously, the controller 3 changes the mode to ‘body in motion’ and the flag to ‘NULL’ (step S110) and proceeds to step S180.

However, if the controller determines that either of the above two conditions are not satisfied at step S100, it proceeds to step S120. At step S120, the controller 3 determines whether the mode is set to ‘body sit still’ and the flag is set to ‘SLIGHT MOVEMENT.’ If both of the above conditions are satisfied (step S120:YES), the controller 3 displays a slight movement starting sign on the display section 34, changes the mode to ‘body in motion,’ and changes the flag to ‘NULL’ (step S130). The controller 3 then proceeds to step S180.

Alternatively, if the controller 3 determines that either of the above two conditions are not satisfied at step S120 (step S120:NO), it proceeds to step S140. At step S140, the controller 3 determines whether the mode is set to ‘body in motion’ and the flag is set to ‘NULL.’ If the above two conditions are satisfied (step S140:YES), the controller 3 displays a body movement ending sign on the display section 34, changes the mode to ‘body sit still’ (step S150), and determines the posture (step S160).

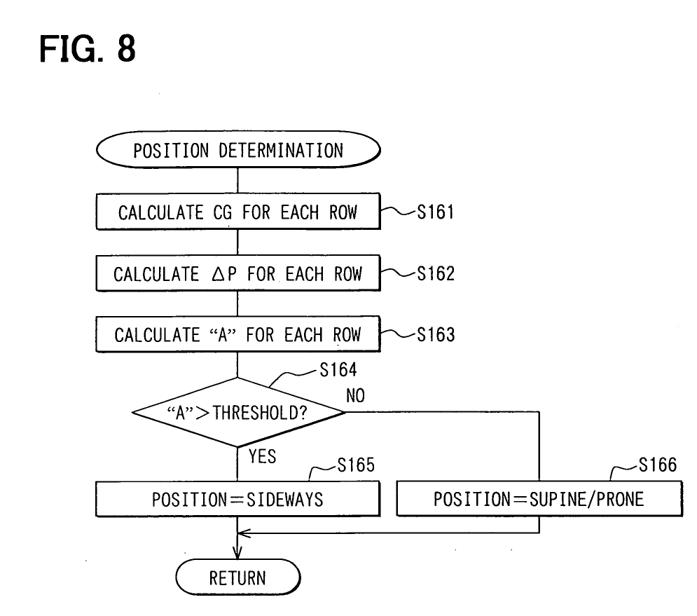

Now, how the controller 3 determines the posture at step 160 is described with reference to FIG. 8.

First, the controller 3 calculates a center of gravity for each row of sensors (step S160). The ‘row’ is defined as being perpendicular to the spine of a person lying normal on the bed 50. The controller 3 then calculates a difference between a pressure taken at a predetermined distance from the center of gravity and an average pressure of a certain row (step S162). Furthermore, the difference of pressures is averaged out to the value A (step S163) and the controller determines whether the value A is larger than a predetermined threshold value (step S164).

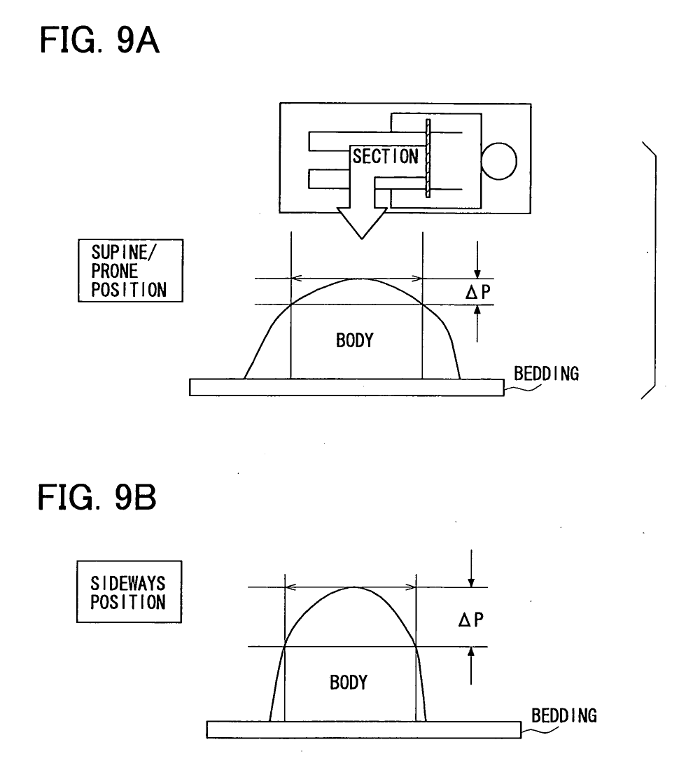

When the controller 3 determines that the value A is larger than the threshold (step S164:YES), it identifies the posture to be a ‘sideways position’ (step S165). Alternatively, when the controller 3 determines that the value A is equal to or lesser than the threshold (step S164:NO), it identifies the posture to be a ‘supine/prone’ position. With reference to FIG. 9, this determination is further described.

FIG. 9A is a graphical diagram of a cross-section of a human torso in a supine/prone position. FIG. 9B is a graphical diagram of a cross-section of a human torso in a sideways position. Because humans tend to have a larger dimension in the lateral direction than in the front-rear direction, a change rate of the pressure distribution according to the lateral arrangement of the pressure sensors automatically differs in the above two cases. Specifically, the supine/prone position presented in FIG. 9A has a relatively gradual pressure change rate and the sideways position illustrated in FIG. 9B has a relatively steep pressure change rate. Based on these different change rates of pressure, an examiner can determine the sleeping position of an examinee. For example, when the value A is larger than a certain threshold, the position can be identified as ‘sideways,’ and when the value A is smaller than the threshold, the position can be identified as ‘supine/prone.’ It should be appreciated that the threshold value itself has to be carefully chosen to make a correct identification.

Upon completing step S165 or step S166 in FIG. 8, the controller 3 completes the position determination routine and proceeds to step S170 of FIG. 5.

At step S170 of FIG. 5, the controller 3 outputs a sleeping posture to the display section 34. That is, in step S170, a body movement ending sign and a sleeping posture at the time of the body movement ending is outputted (step S170). Though the position determination process is executed in parallel as shown in step S160, this is based on the assumption that a position change is always accompanied by body movement.

Next, the controller 3 proceeds to step S180. At step S180, the controller 3 displays respiratory information output and position output on the display section 34. Then, the controller 3 increases the data count by 1 (step S190) and determines whether all the sensor signals are retrieved (step S200). If signal reading has been completed (step S200:YES), the controller 3 terminate the process. If signal reading has not been completed (step S200:NO), the controller 3 returns to step S20.

The outputs from the three parts are now described. The three outputs include body movement information from steps S110, S130, and S150; sleeping posture from step S170; and respiratory information and position from step S180. These are now described with reference to FIG. 10.

FIG. 10 shows respiratory information, body movement information, position, and sleeping posture from the top to the bottom over a period of time on the horizontal axis.

Respiratory information is shown with the depth of respiration as a vertical axis. Body movement information is shown together with the respiratory information. The body movement information is color-coded according to the magnitude of the movement and the period of movement is indicated as the length of the bar in the graph. FIG. 10 shows three periods of body movement. Though the colors of the small squares corresponding to the body movement periods in FIG. 10 are not clearly shown, the body movement period on the left shows yellow to red to blue transition. The period in the center has the same pattern. The body movement period on the right has a pattern of red to blue transition. In the present embodiment, a turnover is shown in red to blue transition of small squares and slight movement is represented by yellow squares. Therefore, FIG. 10 communicates that the body movements on the left and center were slight movement toward turnover transitions. The body movement on the right was a turnover. Further, duration of those body movements can be seen.

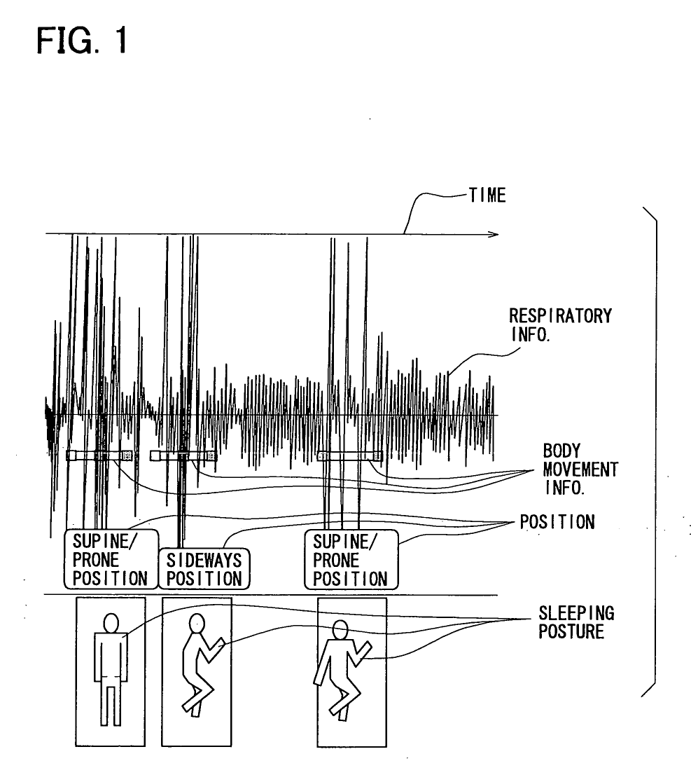

As described above, at the end of body movement, position and sleeping posture are outputted. In FIG. 10, the body movement on the left ended in a supine/prone position. The movement at the center ended in a sideways position. The body movement on the right ended in a supine/prone position. These are all displayed in character for the ease of recognition. Furthermore, a visual display of the sleeping posture is shown by 165 dots. This number 165 is equal to the number of pressure-sensing devices 221 installed in the sensor sheet 2. The strength of pressure detected by the pressure-sensing devices 221 is presented in 6 levels, using 6 colors of gradation in this embodiment. In FIG. 10, although the color of each dot represented by a small circle is lost in the sleeping posture representation, the dots in relatively lighter color correspond to the body area of the examinee. In reality, the sleeping posture representation is assumed to be the one in FIG. 1, with its pressure distribution around the torso shown in the gradation.

In the present embodiment, the pressure-sensing device 221 corresponds to the ‘sensor,’ or the ‘living body information detection means,’ or the ‘sleeping posture detection means.’ The microcomputer 32 and the display section 34 correspond to the ‘display control means’ in the scope of the patent claims. The microcomputer 32 corresponds to the ‘position determination means.’

The living body information display apparatus 1 in the present embodiment can identify the two-dimensional area of a sleeper from signals of pressure-sensing devices 221 that are arranged in rows and columns and yield signals when detecting pressure above a predetermined value. Visual representation of the signals from those devices can intuitively convey a sleeping posture of the sleeper (examinee). In this manner, the examiner can differentiate two supine/prone positions by the position of limbs or two sideways positions by the spinal curvature. The examples in the drawings in FIG. 1 and FIG. 10 show that the body movement periods on the left and on the right (in the graph) both ended up in the supine/prone position, but the left foot supports the right foot at the end of the right body movement period. The result is that the right side of the hip/waist is afloat a little bit and the pressure around the left waist is increased. This kind of condition can only be grasped from the intuitive visual display of the sleeping posture, not from the simple positional information.

Further, as the sleeping posture can be displayed along with the respiratory information and the body movement information, the examiner can grasp the position and posture of the sleeping examinee when the examiner finds a notable change or an abnormality in the living body information. Thus, the examiner can effectively analyze the cause of the problem. This apparatus 1 is especially useful because even an examiner of little experience can easily determine the condition of the examinee.

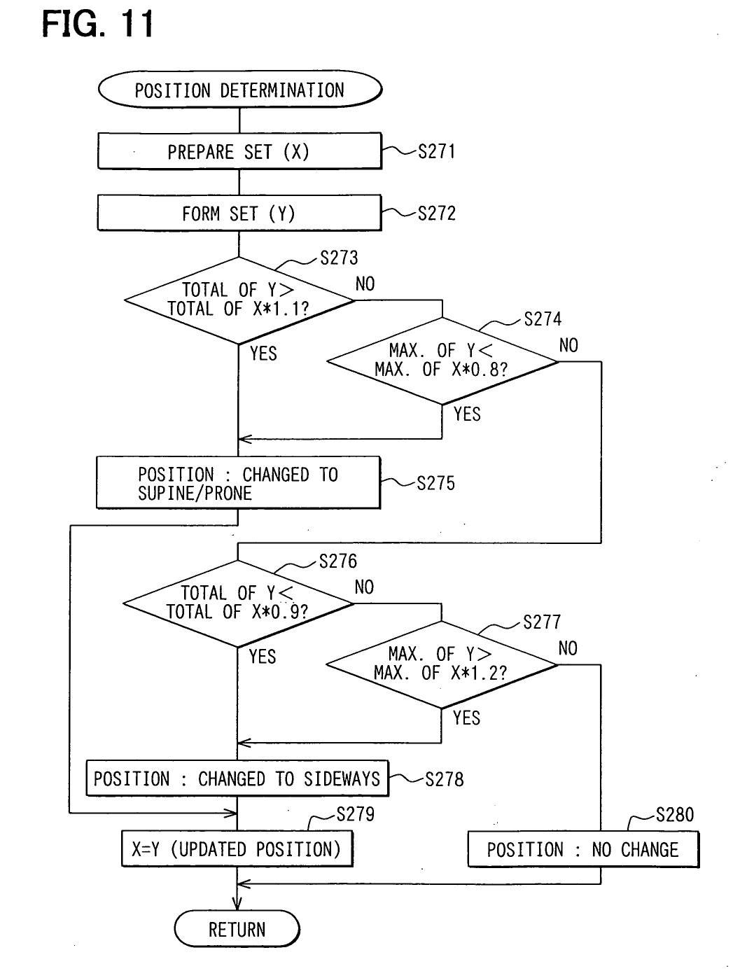

FIG. 11 depicts a second embodiment of a posture determination process according to the principles of the present invention.

The sleeping posture is initially set to be in the supine or prone position, as assumed in the course of the description.

First, the controller 3 retrieves a set(x) of sensor-sensed values (pressure values) that were collected from the pressure-sensing devices 221 under pressure of the sleeper's weight from the memory 33 (step S271). This set(x) contains both the number of pressure-sensing devices 221 that sensed the weight of the sleeper and the pressure values associated therewith. The controller 3 updates this set(x) of sensor-sensed values every time the sleeping posture is determined to have changed at step S279, which will be described later, and stores the updated set(x) in the memory 33.

Next, the controller 3 forms a set(y) of sensor values based on the latest signals from each of the pressure-sensing devices 221 actually sensing the weight of a sleeper (step S272). The controller 3 then multiplies the total number of weight sensing sensors in set(x) by 1.1 and compares them with the total number of weight sensing sensors in set(y) to determine if the total number of sensors in set(y) is greater than the total number of sensors in set(x) (step S273). If the total number of sensors in set(y) is greater than the total number of sensors in set(x) (S273:YES), the controller 3 determines that the sleeping posture has changed from the sideways position to the supine/prone position and memorizes it as such (step S275). Such a determination is reached because the change in the number of signals indicates an increase in the area of bedding that the sleeper's body contacts.

However, if the total number of sensors in set(y) is less than or equal to the total number of sensors in set(x) (S273:NO), the controller 3 proceeds to step S274. At step S274, the controller 3 identifies a sensor that is now sensing the largest signal (the maximum value of set(y)) and a sensor that sensed the largest signal in the past (the maximum value of the set(x)). The controller multiplies the maximum value of set(x) by 0.8 and compares it to the maximum value of set(y).

If the maximum value of set(x) multiplied by 0.8 is greater than the maximum value of set(y) (S274:YES), the controller 3 identifies the sleeping posture to have changed from the sideways position to the supine/prone position. This is because the difference in the maximum signals indicates a great decrease in the weight per unit area on the bedding 60. The controller 3 then proceeds to step S275 and memorizes the sleeping posture as being in the supine/prone position.

However, If the result of the maximum value of set(x) is less than or equal to the maximum value of set(y) (S274:NO), the controller 3 proceeds to step 276. Then, the controller multiples the total number of weight-sensing sensors in the past from set(x) by 0.9 and compares it to the total number of present weight-sensing sensors from set(y). If total number of present weight-sensing sensors from set(y) is less than the total number of weight-sensing sensors in the past from set(x) multiplied by 0.9 (S276:YES), that is, the area of sleeper's body contacting with bedding 60 has decreased, the controller 3 proceeds to step S278 and the controller 3 memorizes the sleeping posture to have changed from the supine/prone position to the sideways position.

However, if total number of present weight-sensing sensors from set(y) is greater than or equal to the total number of weight-sensing sensors in the past from set(x) multiplied by 0.9 (S275:YES), the controller 3 proceeds to step S277. At step 277, the controller 3 multiplies the above mentioned maximum of set(x) by 1.2 and compares it to the maximum of set(y), to determine if the latter is larger than the former. If the maximum of set(y) is greater than the maximum of set(x) multiplied by 1.2 (S277:YES), the controller 3 determines that pressure per unit area in the bedding 60 has increased substantially and the sleeper is identified to have changed sleeping posture from the supine/prone position to the sideways position. The controller 3 then proceeds to step 278 and memorizes the sleeping posture as being sideways.

However, if the maximum of set(y) is less than or equal to the maximum of set(x) multiplied by 1.2 (S277:NO), the controller 3 proceeds to step S280 and memorizes the sleeping posture as having not-changed.

Furthermore, at step S275 or S278, when a change in the sleeping posture is memorized, the controller 3 proceeds to step S279. At step 279, the controller 3 overwrites the past set(x) with the pressure-sensing devices 221 being under actual pressure (being under the sleeper) according to the latest sleeping posture set(y).

It should be appreciated that the controller 3 determines the examinee's position as being the supine/prone position or the sideways position at different times and occasions. The determination is based on a change in the area of pressure derived from the pressure sensors and a change rate of the maximum pressure (weight) of the examinee. Generally speaking, a human torso is larger in the lateral direction than in the front-rear direction. As a result, the area of pressure against the bedding is different depending on the examinee's position—supine/prone position or sideways position. Therefore, a change in pressure area against the bedding can be used to determine an examinee's transition between positions. Also, when the determination is based solely on the change of the area, the change rate of the maximum pressure is also taken into account. That is, when the position is transitioning from the supine/prone position to the sideways position, the pressure per unit area increases and the maximum value of pressure increases. On the contrary, when the position is transitioning from the sideways position to the supine/prone position, the pressure per unit area decreases and the maximum value of pressure also decreases. The area of the applied pressure and change rate of the maximum pressure are used as indicators to appropriately determine the posture and the position.

Living body information to be displayed is not necessarily limited to the above-mentioned types but may also include pulse waves, thoracoabdominal movements and the like. In the above embodiment, although the living body information display apparatus 1 is realized in a relatively simple structure, it is possible to detect a brain wave, for example, as living body information. Additionally, sleeping posture images may be taken by an infrared camera or the like and displayed in synchronization with the living body information. This complexity is only capable when a complicated and expensive apparatus is provided. There are, between the simple apparatus and the complicated one, trade-offs in terms of reality of captured information and the cost. However, if the above-described structure is pursued, the structure becomes simple and sleeping postures can be appropriately and effectively grasped.

In the above embodiment, while only an upper body is displayed as a sleeping posture, the whole body of a sleeper may be displayed. In the above embodiment only an upper body is displayed because it seems like the upper body has a major effect on living body information such as respiratory abnormalities and the like. However, displaying sleeping posture as a whole body is an effective way to intuitively understand the situation. Also, in FIG. 10, though sleeping postures are depicted by the color-coded pressure values picked up by the pressure-sensing devices 221, the sleeping postures can also be represented by outlines as shown in FIG. 1.

While the sleeping posture is displayed at the end of each body movement in the above embodiment, it may be displayed in continuation periodically. However, the same posture is usually continued for a certain period of time and, therefore it is beneficial to display the sleeping posture only at the time of a specific change and/or abnormality in the living body information. This lowers the process load and helps the examiner. For example, if the posture is displayed in continuation, a difference between the former observation and the current one must be picked up by the examiner for himself/herself. Such judgment is not necessary if the posture is displayed only when a noticeable change and/or abnormality occurs.

Though the sleeping posture is displayed in two dimensions in the above embodiment, it is possible to display the sleeping posture in three dimensions, if a three-dimensional detector for sleeping posture is adopted.

In the above embodiment, a pressure-sensing device 221 is used only as an example for the sensors. However, a vibration sensor may substitute the pressure-sensing device 221. In that case, the vibration sensor can be made with piezo-film element, a PVDF element or the like.

Though pressure signals from the pressure-sensing devices 221 contribute to both sleeping posture determination and living body information detection in the above embodiment, it is possible to detect living body information by using other types of sensors. However, the above embodiment makes it possible to realize a very simple structure of the apparatus.

1. A method for displaying living body information comprising:
arranging a plurality of sensors under a living body in rows and columns approximately perpendicular and parallel to a longitudinal direction of the living body, and positioning the sensors in predetermined intervals;
outputting signals from the plurality of sensors, the signals corresponding to at least one of a pressure and variation of pressure of the living body;
detecting and outputting the living body information of the living body;
obtaining the signals from the plurality of sensors, processing the signals to display a difference of the at least one of the pressure and variation of pressure in a color coded manner, and outputting processed signals providing a posture of the living body in the color coded manner;
outputting the processed signals at an end of each body movement over a period of time, and outputting the living body information over the same period of time as the posture of the living body; and
displaying the posture of the living body and the living body information.

2. The method according to claim 1, wherein
the detecting and outputting the living body information includes:
obtaining the signals from the sensors;
converting the signals to the living body information; and
outputting converted signals.

3. The method according to claim 2, wherein
the living body information includes at least one or more of a respiration, a body movement, a pulse, a thoracoabdominal movement, and a position of the living body.

4. The method according to claim 1, wherein
in the processing the signals, the signals are processed in order to display only a portion of the living body as the posture including an upper body of the living body, and
the outputting the processed signals providing only the portion of the living body in the color coded manner.

5. The method according to claim 1, wherein the processing the signals is performed only when a notable change or an abnormality occurs in the living body information so that the displaying the posture of the living body is performed only when the notable change or the abnormality occurs in the living body information.

6. A method for displaying living body information comprising:
arranging a plurality of sensors under a living body in rows and columns approximately perpendicular and parallel to a longitudinal direction of the living body, and positioning the sensors in predetermined intervals;
outputting signals from the plurality of sensors, the signals corresponding to at least one of a pressure and variation of pressure of the living body;
obtaining the signals from the plurality of sensors, processing the signals to display a difference of the at least one of the pressure and variation of pressure in a color coded manner, and outputting processed signals providing a posture of the living body in the color coded manner;
determining whether the living body is in one of a supine/prone position and a sideways position based on the signals and also a change rate of the at least one of the pressure and the variation of pressure across the rows of the sensors;
outputting the processed signals at an end of each body movement over a period of time, and outputting the living body information of a living body position over the same period of time as the posture of the living body, wherein the living body position is obtained in the determining; and
displaying the posture of the living body and the living body information.

7. The method according to claim 6, wherein
in the determining, the living body position is determined in the supine/prone position when another difference between a maximum value among the at least one of the pressure and the variation of pressure in a certain row of the sensors and at least another pressure and variation of pressure derived at a predetermined distance from where the maximum value is derived is smaller than a predetermined value, and
the living body position is determined in the sideways position when the difference is greater than the predetermined value.

8. The method according to claim 7, wherein
in the determining, the living body position is determined in the supine/prone position when an average of the differences across multiple rows is smaller than the predetermined value,
the living body position is determined in the sideways position when the average of differences is greater than the predetermined value, and
the differences are calculated in each row between the maximum value of the at least one of the pressure and variation of pressure and the corresponding value derived at the predetermined distance from where the maximum value was derived.

9. A method for displaying living body information comprising:
arranging a plurality of sensors under a living body in rows and columns approximately perpendicular and parallel to a longitudinal direction of the living body, and positioning the sensors in predetermined intervals;
outputting signals from the plurality of sensors, the signals corresponding to at least one of a pressure and variation of pressure of the living body;
obtaining the signals from the plurality of sensors, processing the signals to display a difference of the at least one of the pressure and variation of pressure in a color coded manner, and outputting processed signals providing a posture of the living body in the color coded manner;
determining whether the living body is in one of a supine/prone position and a sideways position based on a change rate of an area where the at least one of the pressure and the variation of pressure is being applied and on a change rate of a maximum value of the at least one of the pressure and the variation of pressure, wherein the determining is made for different durations and at different occasions;
outputting the processed signals at an end of each body movement over a period of time, and outputting the living body information of a living body position over the same period of time as the posture of the living body, wherein the living body position is obtained in the determining; and
displaying the posture of the living body and the living body information.

10. The method according to claim 9, wherein
in the determining, the living body position is determined in the supine/prone position when a change of the area where the at least one of the pressure and the variation of pressure is being applied is greater than a predetermined value, and
the living body position is determined in the sideways position when the change of the area is smaller than the predetermined value.

11. The method according to claim 9, wherein
in the determining, the living body position is determined in the supine/prone position when a change of the area where the at least one of the pressure and the variation of pressure is being applied is greater than a predetermined value and when the change rate of the maximum value of the at least one of the pressure and the variation of pressure is smaller than another predetermined value, and
the living body position is determined in the sideways position when the change of the area is smaller than the predetermined value and when the change rate of the maximum value is greater than the another predetermined value.
